(19) United States
(12) Patent Application Publication
(10) Pub. No.: US 2007/0254367 A1
(43) Pub. Date: Nov. 1, 2007

(54) Methods for extending the life span of marine invertebrate cells

(76) Inventors: Shirley A. Pomponi, Ft. Pierce, FL (US); Jane Thompson, Vero Beach, FL (US); James J. Bottesch, Rockledge, FL (US); Susan H. Sennett, Sebastian, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/796,862

(22) Filed: Apr. 30, 2007

Related U.S. Application Data
(60) Provisional application No. 60/796,251, filed on Apr. 28, 2006.

(51) Int. Cl. C12N 15/86; C12N 5/06

(52) U.S. Cl. 435/456; 435/325

(57) ABSTRACT

The subject invention provides materials and methods for extending the life span and/or immortalizing marine invertebrate cell lines. Specifically exemplified herein are methods for extending the life spans for sponges, cnidarians, mollusks and ascidians. In a preferred embodiment exemplified herein, the cells whose lives are extended are marine sponge cells.

### CROSS-REFERENCE TO A RELATED APPLICATION

This application claims the benefit of U.S. Provisional Application Ser. No. 60/796,251, filed Apr. 28, 2006.

### FIELD OF THE INVENTION

The subject invention pertains to the use of viruses and polynucleotide sequences associated with cell survival and proliferation to extend the life span of marine invertebrate cells. It establishes methodology for maximal exploitation of existing knowledge in transforming mammalian cells for application to production of marine invertebrate cell biomass and/or cell metabolites.

### BACKGROUND OF INVENTION

The general interest in sponge-derived biopharmaceuticals has increased substantially ever since the discovery and identification of biomedically relevant secondary metabolites by Bergmann and Feeney in the early 1950s (Bergmann, W; Feeney, R; 1950 J. Am. Chem. Soc. 72:2809-2810; 1951 J. Org. Chem. 16:981-987). An increasing number of sponge-derived compounds of pharmaceutical interest have been discovered, identified, screened and tested in initial clinical trials (Newmann, D; Cragg, G; 2004 J. Nat. Prod. 67:1216-1238; Rawat, D; Joshi, M; Joshi, P; Atheaya, H. 2006, Med. Chem. 6(1)33-40). Unfortunately, these bioactive agents are often produced in trace amounts too low to cover the worldwide need. Munro et al. (Munro, M; Blunt, J; Dumdei, E; Hickford, S; Lill, R; Li, S; Battershill, C; Duckworth, A., 1999 J. Biotech. 70:15-25) perfectly exemplify this shortage by demonstrating that in continuous global-scale melanoma treatment using halichondrin B derived from the sponge Lissodendoryx sp., at least 5000 tons of sponge biomass would be needed, while only 300 tons of biomass of this species are estimated available in the world's oceans.

Harvesting sponges for bioactive components is neither environmentally nor economically feasible, therefore, an alternative strategy for the bulk production of sponge-based pharmaceuticals is essential (Pomponi, 2006, Can. J. Zool. 84:167-174). Large-scale, in vitro sponge cell culture may provide a well-defined and controllable environment for the production of chemicals and other bioproducts of interest. However, all attempts to maintain a normal or immortalized sponge cell line have been unsuccessful to date (see review Rinkevich, 2005 J Biotech 70:133-153). Significant gaps in our understanding of culture conditions for marine invertebrate cells exist. The use of innovative techniques and strategies may provide new insights and progress in the extending the life span of marine invertebrate cells for biotechnological applications.

There have been numerous attempts to develop an in vitro model (i.e., a cell line) as a tool for marine sponge cell research and as a source of marine bioproducts with pharmaceutical application (for review, see Pomponi, S. A. 2006, Can J Zool 84:167-174). While some progress has been made, the ultimate goal of a clonal, axenic, continuously dividing marine sponge cell line has yet to be achieved. Despite this limitation, research is progressing with development of alternative culture systems (Custodio, M. R. et al. 1998, Mech Ageing Dev 105(1-2):45-59; Kreuter, M. H. et al. 1992, Comp. Biochem. Physiol. 101C(1):183-187; Munro M. H. et al. 1999, J Biotechnol 70(1-3):15-25; Muller, W. E. et al. 2000, J Ant Prod 63(8):1077-1081), reports of cell proliferation (Krasko, A. et al. 2002, DNA Cell Biol 21(1):67-80), and elucidation of some of the basic cellular and molecular traits of marine sponge cells (Muller, W. E. et al. 2001, Gene 276(1-2):161-173; Schutze, J. et al. 2001, J Mol Evol 53(4-5):402-415).

One advance is the discovery that marine sponge cells respond to the mitogenic lectin, phytohemagglutinin (PHA) (Pomponi, S. A. and R. Willoughby 1994 “Sponge cell culture for production of bioactive metabolites” In: van Soest, van Kempen, and Braekman, editors. Sponges in Time and Space. Rotterdam: Balkema. p 395-400). Previously, this sponge cell culture phenomenon had been documented only by noting cell numbers, protein content, esterase activity, and DNA content in primary cultures of PHA-treated cells (Willoughby, R. and S. A. Pomponi 2000, “Quantitative assessment of marine sponge cells in vitro: development of improved growth medium” In Vitro Cell Dev Biol—Animal 36:194-200). Even in mammalian cell lines, the molecular basis of the PHA response has been poorly understood, however, it has been demonstrated that PHA stimulation effects specific changes in gene expression in marine sponge cells (Willoughby, R., 2002, In vitro gene expression in marine sponge cells stimulated by phytohemagglutinin, Ph.D. Dissertation, Florida Institute of Technology, Melbourne, Fla.). This evidence was based on a demonstration of broad-scale genetic homology among sponges and higher metazoans.

The Model Sponge

The marine sponge Axinella corrugata (Phylum Porifera, Class Demospongiae, Order Halichondrida, Family Axinellidae) has been used in our laboratory as a model system for marine invertebrate cell culture for more than ten years. It produces the bioactive compound stevensine (Wright, A. E., S. E. Chiles, S. S. Cross 1991, J Nat Prod 54(6):1684-1686) which has antitumor properties (U.S. Pat. No. 4,729,996) and also functions as a neurotransmitter blocker (Coval, S. J. et al. 1996, U.S. patent application Ser. No. 08/644,138). The production of this compound, which is believed to be of sponge origin, makes A. corrugata an appropriate candidate for cell culture studies that focus on biosynthesis as a model for in vitro production of potentially therapeutic products (Andrade, P. et al. 1999, Tetrahedron Lett 40(26):4775-4778). Success in establishing primary cell cultures of this species (Pomponi, S. A., R. Willoughby, and M. Kelly-Borges 1997a, “Sponge Cell Culture” In: Cooksey K, editor. Molecular Approaches to the Study of the Ocean. Chapman & Hall. p 423-429; Pomponi, S. A. et al. 1997b, “Development of techniques for in vitro production of bioactive natural products from marine sponges” In: Invertebrate Cell Culture: Novel Directions and Biotechnology Applications. Maramorosch K, Mitsuhashi J, editors. Science Publishers, Inc. p 231-237; Pomponi, S. A. et al. 1998. “In vitro production of marine-derived antitumor compounds” In: Le Gal Y, Halvorson H O, editors. New Developments in Marine Biotechnology. New York: Plenum Press p 73-76) and in vitro production of stevensine (Pomponi et al. 1997b, 1998 supra) have been demonstrated. In addition, A. corrugata has been used as an in vitro model for the analysis of the effects of culture medium factors on DNA, protein, and esterase activity (Willoughby, R and Pomponi, S. A. 2000 In Vitro Cellular & Developmental Biology, 36:194-200) and for establishing broad-scale genetic homology among sponges and higher metazoans (Willoughby 2002 supra).

Marine Sponge Genes and Gene Expression

Previously, some insight into marine sponge potential for molecular response has been achieved by comparing individual sponge nucleic acid sequences to those of model organisms, thus accomplishing gene discovery by database homology analysis. Many of these previous studies have focused on phylogeny and evolutionary genetics, rather than characterization of in vitro (or even in situ) physiology for functional purposes. Indeed, few have looked at the actual expression of the characterized genes, though a recent contrary trend is evident. In one of the earliest expression studies, Schroder et al. 1988 (J Biol Chem 263(31):16334-16340) used immunoprecipitation to quantify ras expression in marine sponge cells. Biesalski et al. 1992 (Oncogene 7(9):1765-1774) reported down-regulation of a myb-related gene in cells of Geodia cydonium. Also, Pfeifer et al. (1993b, J Cell Sci 106 (Pt 2):545-553) reported increased polyubiquitin expression in response to homologous aggregation factor. These studies employed dissociated sponge cells and were therefore an early look at the function of sponge cells in vitro.

More recent studies, many of which utilize intact sponge tissue or re-aggregated sponge cells, include those by Wiens et al. (2000b, J Mol Evol 50(6):520-531) and Kruse et al. (1999, J Cell Sci 112(part 23):4305-4313), who looked at differential expression in response to allograft rejection in marine sponge tissue. Profilin expression was also up-regulated in the presence of non-self sponge molecules (Muller, W. E. et al. 1999b, DNA Cell Biol 1(12):885-893). Potential self-recognition molecules were up-regulated in autografts, according to Wimmer et al. (1999b, Cell Adhes Commun 7(2):111-1124), Fernandez-Busquets et al. (1998, J Biol Chem 273(45):29545-29553) and Blumbach et al. (1999, Immunogenetics 9(9):751-763). Molecules associated with immune responses were reviewed by Muller et al. (1999c, Transplantation 68(9):1215-1227). Scheffer et al. (1997, Biological J Linnean Soc 61:127-137) used whole sponges to study SRF expression in response to heat stress. Whole sponges were also used to document increased MA-3 expression (Wagner, C. et al. 1998, Mar Biol 131:411-421). Weins et al. (1999a, Tissue Cell 31(2):163-169) reported down-regulation of a putative tumor suppressor in response to cadmium exposure, and Krasko et al. (1999, J Biol Chem 274(44):1524-1530) reported up-regulation of a protein kinase and a potential ethylene-responsive protein in sponge tissues exposed to ethylene. Utilizing intact tissue, Weins et al. (1999c, Marine Biol 133:1-10) documented increased HSP70 and thioredoxin expression in response to 17β-estradiol. Increased HSP70 expression was also noted in response to tributyltin (Batel, R. et al. 1993 Mar Ecol Prog Ser 93:245-251.). Phosphorylation of p38 was detected in sponge primmorphs treated with hypertonic medium (Bohm, M. et al. 2000, Biol Cell 92:95-104). A similar culture system was used to study differential expression of a longevity assurance-like gene (Schroder, H. C. et al. 2000 Mech Devel 95:219-220) as well as collagen and silicatein genes (Krasko, A. et al. 2000 Eur J Biochem 267:4878-4887.). Actual sponge cell cultures (i.e., not explants or primmorphs) were once again used to demonstrate ras up-regulation in response to sponge aggregation factor by Wimmer et al. (1999b, Cell Adhes Commun 7(2):111-1124). Intact sponges stressed by exposure to UV light demonstrated increased expression of an excision repair gene homologue as measured by Northern blot comparisons (Batel, R. et al. 1998 Mutat Res 409(3):123-33.).

Recently, researchers have begun to directly explore sponge functional genetics in relation to that of other organisms. Muller and colleagues have begun to present multiple cases for genetic homology, as well as functional similarities, between sponges and higher organisms (Muller, W. E. et al. 2001, Gene 276(1-2):161-173; Gamulin, V. et al. 2000, Biological Journal—Linnean Society 71( ):821-828; Seack, J. et al. 2001, Biochim Biophys Acta 1520(1):21-34; Bohm, M. et al. 2000, Biol Cell 92:95-104; Wiens, M. et al. 2000a, Cell Death Differ 7(5):461-469; Pahler, S. et al. 1998c, Proc R Soc Lond B Biol Sc. 265(1394):421-425). Willoughby (2002 supra) used cross-species DNA microarray technology to profile gene expression in the sponge Axinella corrugata stimulated by PHA and discovered close homology between many human and sponge gene sequences using this approach. Willoughby (2002 supra) identified specific genes that may be involved in apoptosis signaling and which represent targets for genetic manipulation of the cultures to expand their life spans.

Transformation of Normal Cells

Normal human cells have a limited life span and enter a non-dividing state called replicative senescence. Telomeres shorten throughout the life span of cultured cells (Allsopp et al. 1992 Proc. Natl. Acad. Sci. USA, 89: 10114-10118) and such shortening may trigger such replicative senescence. Human telomerase reverse transcriptase (hTERT), the catalytic subunit of telomerase, is an effective life span extending agent with high efficacy in numerous studies of mammalian cells (Bodnar et al. 1998 Science, 279: 349-352; Yang et al. 1999 J. Biol. Chem., 274(37): 26141-26148; Steinert et al. 2000 Biochem Biophys Res Comm. 273:1095-1098). Introduction of an expression vector containing hTERT elevates the intracellular level of telomerase activity, resulting in cells with an extended life span past replicative senescence. Other studies have demonstrated that some cell types overcome senescence by coexpression of hTERT and human papilloma virus (HPV) (Farwell et al. 2000, Am. J. Path., 156(5):1537-1547; Veldman et al. 2001, i 75(9): 4467-4472) or simian virus 40 (SV40) large T antigen (Krump-Konvalinkova et al. 2001 Laboratory Investigation 81(12): 1717-1727). The life span of normal human cells have also been extended by HPV and transformed into a malignant state by the combination of HPV and ras oncogene (Rhim et al. 1994 Proc Natl Acad Sci USA 91:11874-11878). The Epstein-Barr virus (EBV) extends the lifespan of normal human lymphocytes (Miller et al. 1974 Proc Nat Acad Sci USA 71(10): 4006-4010).

### BRIEF SUMMARY

The subject invention provides materials and methods for extending the life span and/or immortalizing marine invertebrate cell lines. Specifically exemplified herein are methods for extending the life spans for cell lines derived from sponges, cnidarians, mollusks and ascidians. In a preferred embodiment exemplified herein, the cells whose lives are extended are marine sponge cells.

In general, the subject invention provides materials and methods for creating an immortalized marine invertebrate cell line by transforming the cells with one or more polynucleotide sequences associated with cell survival and proliferation. In one embodiment, the subject invention pertains to the immortalization of a sponge cell line by transforming the cells with Epstein Barr virus. A further embodiment for immortalizing the sponge cell line comprises transforming the cells with human telomerase reverse transcriptase.

### BRIEF DESCRIPTION OF DRAWINGS

The file of this patent contains at least one drawing executed in color. Copies of this patent with color drawings(s) will be provided by the Patent and Trademark Office upon request and payment of the necessary fee.

FIG. 1 Agarose gel of Xestospongia muta DNA PCR products to BALF2 (EBV). Lanes 1 & 8: size marker; lanes 2-4: X. muta sponge cells treated with increasing concentrations of EBV (note PCR product for BALF2 in lane 4); lanes 5 & 7: negative control (untreated sponge cells); lane 6: positive control (human lymphoblastoid 1A-2 cells).

FIG. 2 Agarose gel of Axinella corrugata DNA PCR products to hTERT. Lanes 1 & 6: untreated cells; lanes 2 & 7: hTERT-transformed sponge cells; lanes 3 & 8: negative control; lanes 4 & 9: hTERT plasmid (positive control); lane 5: empty; lane 10: size marker. (Different primer sets were used for samples in lanes 2-4 vs those in lanes 7-9.)

FIG. 3 Agarose gel of Axinella corrugata cDNA PCR products to hTERT. Lanes 1 & 6: size marker; lane 2: untreated sponge cells (containing native telomerase reverse transcriptase); lane 3: transformed sponge cells; lane 4: positive control (hTERT plasmid); lane 5: negative control.

FIG. 4 Immunocytochemistry results. The breast cancer line MCF7 is a fast growing, rapidly dividing cancer cell, and thus shows high numbers of the target enzyme (as indicated by the bright red fluorescence) (B1). The non-transformed Axinella corrugata cells present little fluorescence (B2), indicating the baseline hTERT-analog levels in untreated cells. Transformed Axinella corrugata archeocytes display bright red fluorescence (B3), which is a clear indication of the presence of the introduced hTERT enzyme.

### DETAILED DISCLOSURE

The subject invention provides materials and methods for extending the life span and/or immortalizing marine invertebrate cell lines. Specifically, the subject invention provides methods for transforming cells of marine invertebrates to extend their life spans thereby providing a convenient and efficient source for various compounds, including bioactive compounds.

Marine invertebrates are the source of many chemicals with pharmaceutical importance. Bulk supply of these compounds can be achieved through cell cultures of the producing organisms. To date, there are no cell lines of marine organisms that produce bioactive compounds

The subject invention provides methods for extending the life spans for sponges, cnidarians, mollusks and ascidians. In a preferred embodiment exemplified herein, the cells whose lives are extended are marine sponge cells. Specifically exemplified herein is the transformation of Axinella corrugata and Xestospongia muta (Phylum Porifera, Class Demospongiae, Order Haplosclerida, Family Petrosiidae).

Most cell types undergo a limited number of divisons and then enter senescence. The materials and methods of the subject invention can be used to extend the time that a cell undergoes cell divisions by delaying or preventing senescence. Thus, as used herein, an immortalized cell line, or continuous cell culture, is one which has an extended ability for population doubling compared to a cell line that has not been treated according to the subject invention.

In general, the subject invention provides materials and methods for creating an immortalized marine invertebrate cell line by transforming the cells with polynucleotide sequences associated with cell survival and proliferation. In one embodiment, the subject invention pertains to the immortalization of a sponge cell line by transforming the cells with Epstein Barr virus. A further embodiment for immortalizing the sponge cell line comprises transforming the cells with human telomerase reverse transcriptase. Other sequences that can be used include, but are not limited to, Akt, PI₃K, EGFR, STAT3, c-FLIP and other oncogenes. The methods of the subject invention further include embodiments wherein a cell line is transformed with multiple polynucleotide sequences associated with cell survival and/or proliferation. The sequences that may be used according to the subject invention may be be the full length sequences, or portions thereof that retain the ability to enhance cell line survival. Preferably, the sequence used will be at least 50% of the full length sequence, more preferably more than 75% and may also be more than 90% or even 95% of the full length sequence. The skilled artisan would also appreciate that certain variations of the sequences can be used. The sequence used will be at least 50% identical of the corresponding portion of the native sequence, more preferably more than 75% identical and may also be more than 90% or even 95% identical to the wild type sequence.

The immortalized cell lines of the subject invention can be used, for example, to study various cellular mechanisms in assays evaluating the effect of various factors on cell death processes and/or to produce desired products. The desired products may be recombinant products and/or products that are naturally produced by the cells.

In a specific embodiment, the cells are transformed with a polynucleotide sequence that confers on the cells the ability to produce useful compounds. These compounds may be, for example, compounds having therapeutic or other useful bioactive properties. In this embodiment, the polynucleotide sequences may be modified to enhance expression of the desired product in the particular marine invertebrate cell line. The modification may be one which, for example, improves expression by utilizing codons that are preferred by the host cells. Appropriate promoters and/or other regulatory sequences can also be used.

The immortalized cell lines of the subject invention can also be utilized for the long-term production of useful quantities of products that are naturally produed by the cells.

The cells of the subject invention can also be utilized to provide “artificial” marine invertebrate tissues. Appropriate substrates can be used to enhance the stability of such tissues.

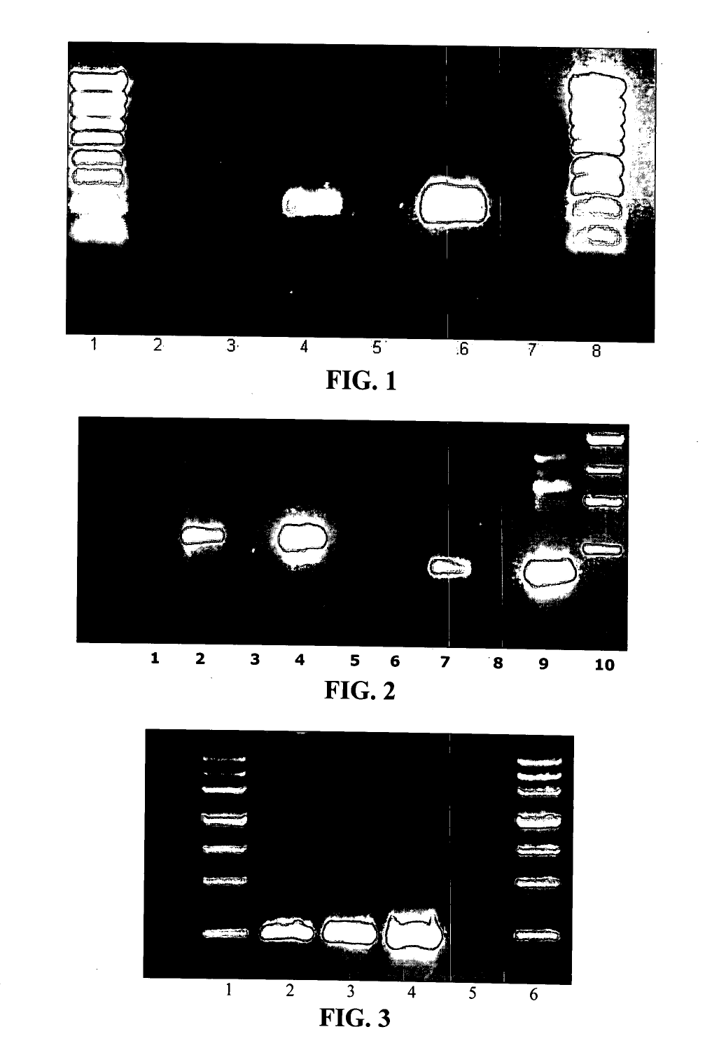

In accordance with the subject invention, marine sponge cells were transfected using two separate transforming agents: Epstein-Barr virus (human herpes virus 4) and human telomerase reverse transcriptase plasmid (hTERT). Cells of Axinella corrugata and Xestospongia muta were exposed to media (RPMI 1640) infected with Epstein-Barr virions from a human lymphoblastoid cell line (1A2, ATCC). Cell cultures were treated in separate experiments with a liposome-mediated transformation reagent (Lipofectamine™) containing human telomerase reverse transcriptase plasmid (hTERT). For both, transfection was optimized by using a concentration gradient of transforming agent to a constant number of cells. Negative controls contained either media without Epstein-Barr virus or reagents without hTERT plasmid. Cells were incubated four to six weeks with a minimum of four complete media changes. DNA was isolated from transformed sponge cells and analyzed for expression of hTERT or BALF2 (DNA binding protein of Epstein-Barr genome) using PCR. Significant PCR products were produced in both Epstein-Barr (FIG. 1) and hTERT transformations (FIGS. 2, 3), which indicates that cell cultures of Axinella corrugata and Xestospongia muta were phenotypically modified by uptake of DNA from Epstein-Barr and hTERT plasmid.

The cell lines produced in accordance with the subject invention can be used, for example, in the production of recombinant proteins and/or secondary metabolites for pharmaceutical, cosmetic, agricultural or other uses.

Following are examples which illustrate procedures for practicing the invention. These examples should not be construed as limiting. All percentages are by weight and all solvent mixture proportions are by volume unless otherwise noted.

### EXAMPLE 1

### Collection of Sponge Source Material

A sample of Axinella corrugata (Phylum: Porifera, Class Demospongiae, Order Halichondrida, Family: Axinellidae) was collected by scuba at a depth of 21.4 m in the Bahamas off Ocean Cay, south of Bimini, (latitude 25°25.72′N, longitude 793°14.55′W). The sponge morphology is erect branching and club-like. It is resilient and fibrous with a corrugated surface and is brick-red in color both external and internal. The sponge was identified as Axinella corrugata as described by Alvarez, van Soest and Rützler (Alvarez, B., R. W. M. van Soest and K. Rützler 1998. A revision of Axinellidae (Porifera: Demospongiae) of the Central West Atlantic region. Smithsonian Contr. Zool. 598: 1-47). A reference sample preserved in ethanol has been deposited in the Harbor Branch Oceanographic Museum (catalog number 003:01034, DBMR number 6-IV-05-3-001) and is available for taxonomic evaluation by those skilled in the art.

A sample of Xestospongia muta (Phylum: Porifera, Class Demospongiae, Order Haplosclerida, Family: Petrosiidae) was collected by scuba at a depth of 21.4 m in the Bahamas off Ocean Cay, south of Bimini, (latitude 25°25.72′ N, longitude 793°14.55′ W). The sponge morphology is vase-like in shape, purple-maroon in external color and tan in internal color. The sponge was identified as Xestospongia muta as described by van Soest (Soest, R. W. M. van. 1980. Marine sponges from Curaçao and other Caribbean localities. Part II. Haploscierida. Studies Fauna Curaçao Caribb. Isl. 62 (191): 1-173). A reference sample preserved in ethanol has been deposited in the Harbor Branch Oceanographic Museum (catalog number 003:01033, DBMR number 6-IV-05-3-003) and is available for taxonomic evaluation by those skilled in the art.

### EXAMPLE 2

### Cell Dissociation, Cryopreservation and Culture

Sponge Cell Dissociation and Cryopreservation: Sponges, maintained in seawater, were cut into pieces (2-5 mm) and placed in cold (4° C.) calcium- and magnesium-free artificial sea water (CMF-ASW) (Spiegel, M. and N. A. Rubenstein. 1972, Exp. Cell Res. 70:423-430). Pieces were minced and filtered through sterile gauze by gentle squeezing. The resulting sponge cell suspension was filtered through a series of 100, 70 and 40 micron cell strainersand concentrated by centrifugation at 250×g for 10 minutes (Sorvall Model RT 6000B, 1000 rpm). Cells were cryopreserved for storage by resuspending the cell pellets in 5 ml cryomedium A (Medium 199/Hanks salts, adjusted to 1000 mOsm). The concentration of suspended cells was determined by cell counting using a hemocytometer. Cell suspensions were diluted in an equal volume of cryomedium B (Medium 199/Hanks salts 45%, FBS 40% and DMSO 15%, adjusted to 1000 mOsm), aliquoted (1 ml) into cryotubes, and placed in a “slow freeze” container at minus 72 degrees Celsius. Cell counts ranged from 1×10⁷ to 1×10⁹ per ml and viability was greater than 93%.

Sponge Cell Culture: Prior to experiments, cryopreserved heterogeneous cell suspensions were reanimated from cryopreservation by quickly thawing using a 50° C. waterbath, and washed with CMF-ASW to remove the cryoprotectant. Next, washed and dissociated crude cell suspensions were semi-enriched for archaeocytes by Percoll™ density gradient centrifugation. A prespun, discontinuous 15-90% Percoll gradient was used (15 ml tube, 2×3 ml Percoll dilutions in CMF-ASW). Raw (i.e., heterogeneous) cell suspensions were carefully layered on top of the Percoll™ column and centrifuged for 10 minutes at 600×g (Sorvall RT6000B centrifuge); the enriched archaeocyte band at the specific Percoll™ interface (as determined by microscopic examination of each cell fraction) was collected using sterile pipettes. Enriched cell fractions were rinsed with CMF-ASW and centrifuged for 10 minutes at 600×g. All cell handling was performed at 4° C. on ice.

Cells were seeded into 6-well plates at 2-5×10⁶ cells per ml in Medium 199 with Earle's salts without L-glutamine, sodium bicarbonate and phenol red. Osmolality of all media used in sponge cell culture experiments was increased to 1000 mOsm using NaCl. The culture medium was supplemented with KH₂PO₄ (60 mg/l), NaCl (1.2 g/l), Na₂HPO₄ (47.9 mg/l), L-glutamine (25 mg/l), and 10% fetal bovine serum. Sponge cells were grown at 22° C. in an environmental chamber (Lab-Line Environ-Shaker 3597-PR).

### EXAMPLE 3

### Transformation of Sponge Cells with Epstein Barr Virus (EBV)

Cells of Xestospongia muta (prepared as described above) were seeded at 1×10⁷ cells/ml into T-25 plates containing varying concentrations of EBV-infected media (5 mls sponge suspension to 1, 2, or 3 mls of EBV media) from a human lymphoblastoid cell line 1A2 infected with EBV (ATCC CRL-8119) (Current Protocols in Immunology. 1991. J. Coligan, A. M. Kruisbeck et. al., eds. John Wiley & Sons). Sponge cells not exposed to EBV were used as the negative control. Cells were incubated for 2 hours at room temperature under low light conditions, transferred to T-25 flasks with standard sponge cell culture medium (Willoughby and Pomponi 2000 supra), and incubated at 22-25° C. for four to six weeks with four complete changes of media, including a wash step. After incubation, DNA from control and transformed sponge cells was extracted using the MasterPure™ Complete DNA/RNA Purification kit (Epicentre, Madison, Wis.). Isolated DNA was analyzed by PCR for expression of BALF2 (the major DNA binding protein of the Epstein-Barr virus). Results show a significant PCR product for BALF2 in X. muta sponge cells treated with the highest concentration of EBV (5 mls sponge suspension: 3 mls EBV media) (FIG. 1).

### EXAMPLE 4

### Transformation of Sponge Cells with Human Telomerase Reverse Transcriptase (hTERT)

The expression vector for human telomerase reverse transcriptase (hTERT) pGRN145 (ATCC MBA-141) was grown in E. coli GC10 (ATCC, Manassas, Va.). Plasmids were harvested using a miniprep kit (Qiagen, Valencia, Calif.). Integrity of the plasmids and the insert were tested using a 1.2% agarose gel and EcoR1 restriction digestion. Sponge cells were transformed using the commercially available transfecting agent Lipofectamine™ 2000 (Invitrogen, Carlsbad, Calif.). All transformation procedures were done at room temperature. For every transformation experiment 234 μl of RPMI medium was incubated for 5 minutes with the transfection agent. Simultaneously, 242 μl of RPMI was incubated with 4 μg of the expression vector. All media used for sponge cell culture and transformations was adjusted to 1000 mOsm with NaCl. Transfection agent and vector were combined and incubated for 20 minutes. Sponge cells were incubated in the hTERT-Lipofectamine™ transfection mix at varying concentrations. Varying amounts of plasmid-Lipofectamine™ were used to a constant number of cells, according to manufacturer protocol (Invitrogen™). Negative controls contained reagents but lacked plasmid DNA. Cells were incubated in serum-free RPMI 1640 for 4 hours under low light conditions, washed and placed in 6-well plates in sponge cell culture medium (Willoughby and Pomponi 2000 supra). Cultures were maintained for four to six weeks at 22-25° C. with a minimum of four complete changes of media including a wash step.

DNA Extraction and Verification: DNA from transformed sponge cells was harvested using the MasterPure™ Complete DNA/RNA Purification kit (Epicentre, Madison, Wis.). Isolated DNA was analyzed for hTERT expression using PCR (PTC-150 MiniCycler, MJ Research/Biorad, Hercules Calif.) with hTERT specific primers as described by Yang et al (1999 J Biol Chem 274(37):26141-26148) (Operon Technologies Inc. Huntsville, Ala.). PCR samples were subjected to an initial denaturing cycle at 95° C. for 180 s followed by 30 elongation cycles consisting of 94° C. for 45 s, 57° C. for 60 s, 72° C. for 90 s, and a final extension cycle of 72° C. for 300 s and were held at 4° C. Gel electrophoresis with a 1.2% agarose gel was used to confirm PCR results. To visualize PCR products a Stratagene Eagle Eye mini Darkroom Cabinet and Stratagene Eagle Sight software, V3.21 were used (Stratagene, La Jolla, Calif.). Results show significant PCR products for hTERT in transformed Axinella corrugata cells and the positive control (FIG. 2). Non-transformed control cells and the negative control did not have a positive PCR product for hTERT. PCR positive and negative controls were successful and PCR amplicons are of the correct anticipated length. RNA Extraction and Verification: To further confirm transformation success, cDNA synthesized from total RNA isolated from a transformed Axinella corrugata cell suspension was examined for the presence of hTERT transcripts. RNA was isolated using the protocol described by Chomczynski and Sacchi (1987 Anal. Biochem. 162:156-159). Complimentary DNA (cDNA) was made from the isolated total RNA using Monsterscript 1^{st}-strand cDNA synthesis kit and standard Oligo dT primers (Epicentre, Madison, Wis.). Samples were subjected to a standard RT-PCR protocol consisting of a 5 min. 40° C. incubation followed by a 40 min., 60° C. incubation. Results were confirmed using 1.2% agarose gel electrophoresis. PCR was performed as before using synthesized cDNA as the PCR template for hTERT transcription analysis. Transformed cells (FIG. 3, lane 3) show a higher amount of hTERT transcripts than in control cells (FIG. 3, lane 2), however, the untreated controls do have native telomerase reverse transcriptase, which is confirmed by immocyctochemisty (see below).

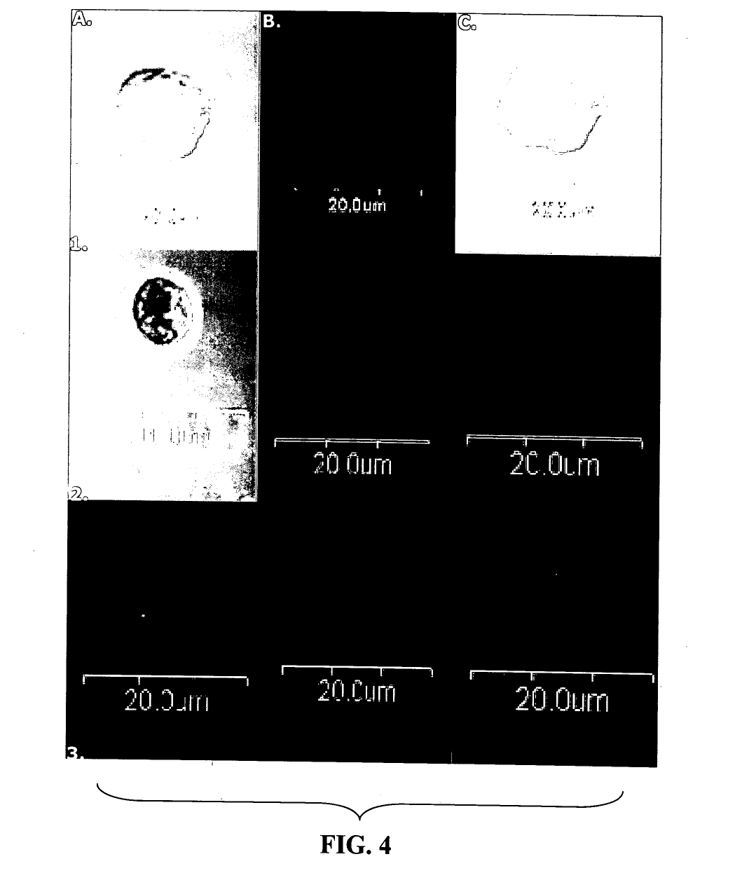

Immunocytochemistry: Transformed Axinella corrugata cell suspensions were further analyzed for hTERT expression vector presence using a custom designed immuno-assay to visualize which types of cells were transformed. Microscopic slides were coated with Poly-L-lysine hydrobromide (0.5 mg/ml) (Sigma, Saint Louis, Mo.) for 1 hour at room temperature, rinsed in ddH₂O and airdried. 10 μl samples of transformed sponge cells, normal (i.e., non-transformed) sponge cells as a negative control, and positive control breast cancer cells were put on coated slides and left to air dry. Next, the dried sponge cells were fixed for 10 minutes using 3.7% paraformaldehyde in CMF-ASW and then permeabilized for 5 minutes using 2% Triton in CMF-ASW. Positive control breast cancer cells were fixed for 5 minutes using 3.7% paraformaldehyde in Dulbecco's phosphate buffered saline (DPBS) followed by permeabilization in a 2% Triton/DPBS solution. All samples were rinsed twice in CMF-ASW, incubated with 1:1000 anti-hTERT rabbit primary antibody (Calbiochem, San Diego, Calif.), for 45 minutes followed by incubation with 1:1000 secondary anti-rabbit IgG, TRITC conjugated antibody (Sigma, Saint Louis, Mo.), for 45 minutes, after which all samples were double rinsed in ddH₂O and air dried. One drop of anti-fade reagent (Molecular probes, Oregon, USA) was added to each cell spot and covered with sterile coverslips, then sealed with clear nail polish. An Olympus FlowView confocal microscope system equipped with a krypton laser was used for the visualization and recording results. FIG. 4 illustrates the results of the immunocytochemistry. The breast cancer line MCF7 is a fast growing, rapidly dividing cancer cell, and thus shows high numbers of the target enzyme (as indicated by the bright red fluorescence, FIG. 4B1). The non-transformed Axinella corrugata cells present little fluorescence, indicating the baseline hTERT-analog levels in untreated cells (FIG. 4B2). Transformed Axinella corrugata archeocytes display bright red fluorescence (FIG. 4B3), which is a clear indication of the presence of the introduced hTERT enzyme.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

We claim:

1. A method for extending the life span of a marine invertebrate cell line wherein said method comprises treatment of an in vitro culture of marine sponge cells with a polynucleotide sequence associated with cell survival and proliferation.

2. The method, according to claim 1, wherein said polynucleotide sequence is a telomerase reverse transcriptase (TERT).

3. The method, according to claim 2, wherein said polynucleotide sequence is human telomerase reverse transcriptase (hTERT).

4. The method, according to claim 1, wherein said polynucleotide sequence is an Epstein Barr virus sequence.

5. The method, according to claim 1, wherein the sponge is Axinella corrugata.

6. The method, according to claim 1, wherein the sponge is Xestospongia muta.

7. A method for extending the life span of a marine invertebrate cell line wherein said method comprises transforming said cell line with a first polynucleotide sequence associated with cell survival and/or proliferation.

8. The method, according to claim 7, wherein said polynucleotide sequence is a telomerase reverse transcriptase (TERT).

9. The method, according to claim 8, wherein said first polynucleotide sequence is human telomerase reverse transcriptase (hTERT).

10. The method, according to claim 7, wherein said first polynucleotide sequence is an Epstein Barr virus sequence.

11. The method, according to claim 7, wherein the cells are cells of the sponge Axinella corrugata.

12. The method, according to claim 7, wherein the cells are cells of the sponge Xestospongia muta.

13. The method, according to claim 1, wherein said method comprises transforming said cell line with multiple polynucleotide sequences associated with cell survival and/or proliferation.

14. The method, according to claim 13, wherein the polynucleotides include at least two polynucleotides selected from the group consisting of hTERT, Epstein Barr Virus, Ras, Akt, PI₃K, EGFR, STAT3, c-FLIP and other oncogenes.

15. A method for producing a useful product from a marine invertebrate cell line wherein said method comprises extending the life span of said cell line by transforming it with a first polynucleotide sequence associated with cell survival and/or proliferation, wherein said method further comprises growing said cell line in a cell culture medium and collecting said product from said cells and/or said medium.

16. The method, according to claim 15, wherein the cells of said cell line have been transformed with a second polynucleotide sequence whose expression results in the production by said cells of said useful product.

17. The method, according to claim 15, wherein said useful product is produced naturally by said cell line.

18. The method, according to claim 15, wherein said first polynucleotide sequence is a telomerase reverse transcriptase (TERT).

19. The method, according to claim 18, wherein said first polynucleotide sequence is human telomerase reverse transcriptase (hTERT).

20. The method, according to claim 15, wherein said first polynucleotide sequence is an Epstein Barr virus sequence.

21. The method, according to claim 15, wherein the cells are cells of the sponge Axinella corrugata.

22. The method, according to claim 15, wherein the cells are cells of the sponge Xestospongia muta.

23. The method, according to claim 15, wherein said method comprises transforming said cell line with multiple polynucleotide sequences associated with cell survival and/or proliferation.

24. The method, according to claim 23, wherein the polynucleotides include at least two polynucleotides selected from the group consisting of hTERT, Epstein Barr Virus, Ras, Akt, PI₃K, EGFR, STAT3, c-FLIP and other oncogenes.

25. A marine invertebrate cell line comprising one or more cells that have been transformed by a first polynucleotide sequence, wherein said first polynucleotide sequence is associated with cell survival and/or proliferation.

26. The marine invertebrate cell line, according to claim 25, comprising one or more cells that have been transformed with a second polynucleotide sequence wherein said second polynucleotide sequence encodes a useful product.

27. The marine invertebrate cell line, according to claim 25, wherein said useful product is a protein that can be used for the prevention, treatment and/or diagnosis of a disease.

28. The marine invertebrate cell line, according to claim 25, wherein said first polynucleotide sequence is a telomerase reverse transcriptase (TERT).

29. The marine invertebrate cell line, according to claim 28, wherein said first polynucleotide sequence is human telomerase reverse transcriptase (hTERT).

30. The marine invertebrate cell line, according to claim 25, wherein said first polynucleotide sequence is an Epstein Barr virus sequence.

31. The marine invertebrate cell line, according to claim 25, wherein the cells are cells of the sponge Axinella corrugata.

32. The marine invertebrate cell line, according to claim 25, wherein the cells are cells of the sponge Xestospongia muta.

33. The marine invertebrate cell line, according to claim 25, wherein said said cell line has been transformed with multiple polynucleotide sequences associated with cell survival and/or proliferation.

34. The marine invertebrate cell line, according to claim 33, wherein the polynucleotides include at least two polynucleotides selected from the group consisting of hTERT, Epstein Barr Virus, Ras, Akt, PI₃K, EGFR, STAT3, c-FLIP and other oncogenes.
